(19) United States
(12) Patent Application Publication
(10) Pub. No.: US 2006/0140874 A1
(43) Pub. Date: Jun. 29, 2006

(54) Stable aerosol formulations of peptides and protein with non-cfc propellants

(76) Inventors: Marc Barry Brown, Watford (GB); Stuart Allen Jones, London (GB); Gary Peter Martin, East Sussex (GB); Yong-Hong Liao, Beijing (CN)

Correspondence Address: FISH & RICHARDSON PC, P.O. BOX 1022, MINNEAPOLIS, MN, 55440-1022, US

(21) Appl. No.: 10/534,634

(22) PCT Filed: Nov. 10, 2003
(86) PCT No.: PCT/GB03/04836

§ 371 Date: Nov. 9, 2005

(30) Foreign Application Priority Data
Nov. 11, 2002 (GB) 0226274.9

(51) Int. Cl. A61L 9/04; A61K 38/28; A61K 9/14; A61K 31/727; A61K 38/47; A61K 38/48; A61K 38/21; A61K 39/395

(52) U.S. Cl. 424/46; 424/85.1; 424/85.4; 424/130.1; 424/94.63; 514/3; 514/2; 514/56; 424/94.61

(57) ABSTRACT

Glycosidicaily stabilised macromolecules, such as proteins and peptides, have substantially greater stability in the presence of hydrofluoroalkane propellants for dispensing from metered dose inhalers, when formulated with polyhydroxylated polyalkenes such as PVA.

The present invention relates to glycosidically stabilised preparations of therapeutic materials for use in metered dose inhalation devices, and methods for their preparation.

Pulmonary delivery has been employed for many years for drugs intended to have localised, rather than systemic, effects. Essentially, there are three types of device available for pulmonary delivery, and these are nebulisers, metered dose inhalers (MDI) and dry powder inhalers (DPI). Each of these has its benefits and its drawbacks.

Nebulisers are particularly effective for the administration of aqueous formulations of drug to non-ambulatory patients. Drug solution is converted into microdroplets which are inhaled by the patient, these microdroplets providing the facility to deliver the drug in a variety of dose volumes, ranging from several milligrams to grams. However, nebulisers are generally large and unsuitable for ambulatory use, and there is a problem with the potential instability of drugs in aqueous solution, as well as during the process of nebulisation. In addition, reproducible dosing can be difficult with these devices.

MDI's are the most widely used pharmaceutical inhalation devices. The formulations used in these devices routinely comprise drug, propellants, and stabilising excipients. In general, the drug is formulated together with the excipients and then combined with the propellants, under pressure, to form either a suspension or solution formulation. Fine, respirable particles of drug are then produced as a consequence of the break up of droplets expelled from the device under pressure, followed by extremely rapid evaporation of the propellants. The amount of drug is controlled by delivering a pre-metered volume of propellant/drug mixture.

The suitability of MDI's to deliver peptide and protein pharmaceuticals has not been well established, and there are concerns for the physical and chemical stability of formulated proteins and peptide particles in propellant mixtures. For these reasons, and the ability to deliver more substantial quantities, DPI's have been generally preferred for the initial research into pulmonary delivery of proteins and peptides.

However, unlike MDI's, the ergonomics of DPI's are manufacture-dependent and, as a result, this can cause confusion amongst patients, which can lead to poor efficacy of therapy. In one study, 40% of patients who had been taught how to use a Turbuhaler®, and who had used it for between 8 months to 8 years, used it so poorly that it was unlikely that the patients were obtaining any therapeutic benefit from the inhaled drugs.

In addition, where the amount of drug to be delivered is not an issue, then the benefits of using DPI's over MDI's is equivocal. In recent studies, there was no evidence that DPI's were any more effective in delivering corticosteroids and β-2 agonist bronchodilators in asthma than MDI's

Furthermore, the aerodynamic performance of MDI and DPI devices containing the same glucocorticoid was compared in vitro, and it was established that the fine particle mass (FPM) delivered by the DPI was flow rate dependent and significantly lower than that achieved using the MDI.

Thus, the primary advantage of DPI's lies in their ability to dispense large quantities of drug from a stable, powder formulation. By contrast, MDI's are able to dispense formulation in a more controlled, and more effective manner, but are more susceptible to physical instability changes. A loss of physical stability can lead to particle aggregation and a lowering in the respirable fraction, or both.

MDI's are propellant-based delivery systems which, until recently, relied on the use of chlorofluorocarbons, or CFC's [trichlorofluoromethane (CFC-11) dichlorofluoromethane (CFC-12) and 1,2-dichlorotetrafluoroethane (CFC-114)], in varying ratios, as the principal component of the formulation. With the universal, phased withdrawal of the use of CFC's, the only two propellants currently approved for inhalation are tetrafluoroethane (HFA-134a) and heptafluoropropane (HFA-227). Both of these hydrofluoroalkanes have boiling points substantially below 0° C., unlike CFC-11 (23.8° C.). In addition, the HFA's have poor solvency for those surfactants commonly employed as excipients in CFC-based MDI's, thereby further complicating the formulation design.

To date, the two most commonly employed formulation strategies for new BFA based MDI's include either the addition of a co-solvent, such as ethanol, to generate a solution MDI, or the incorporation of novel stabilising excipients that are soluble in HFA's to form a suspension MDI. Addition of a co-solvent to a drug-propellant mix can enhance the solubility of the drug to a point where it is completely dissolved in the BHA vehicle. As a consequence, a solution MDI generates respirable particles in a different manner to more traditional suspension formulations. Within a suspension MDI, particles of a defined size have already been manufactured and simply require safe storage and delivery by the device. However, a solution uses the design of the device and the energy created by the evaporating solvent to form the particles upon actuation of the metering valve. The size of the particles ejected from a solution MDI is, therefore, heavily dependent on the actuation orifice diameter and the device design (Lewis et al., 1998). Several research groups have demonstrated that optimisation of these two parameters can potentially produce a dramatic increase in the delivery efficiency of the MDI compared to suspension based formulations (LeBelle et al., 1996;Stein, 1999).

There are, however, several fundamental flaws with formulating an MDI as a solution, including: lack of specific drug targeting; reduced chemical and physical stability; and, a loss of control over the dissolution rate (Leach et al., 2002). The lack of control over the specific targeting within the deep lung has recently been studied by Hochhaus et al. This group described mean pulmonary residence time as the major influence on pulmonary targeting of steroids. They showed that solution MDI's had a much lower pulmonary resonance time compared to suspension formulations and suggested that this could result in a lack of lung steroid receptor specificity, hence an increased chance of side effects (Hochhaus et al., 1998). However, by far the most difficult problem to overcome when manufacturing solution MDI's the reduction in the chemical stability of the drug (Sonie et al., 1992).

Blondino and Byron investigated the effects of a solution formulation on the chemical stability of a model drug acetylsalicyclic acid (Blondino and Byron, 1998). Results from this work indicated that inclusion of a co-solvent to enhance the drug-excipient-propellant compatibility also increased the chemical degradation of the drug. In this study, this was found to be dependent on the concentration of surfactanti Furthermore, within a solution formulation, the drug is exposed to the significant levels of dissolved water taken up in the HFA propellant (Vervaet and Byron, 1999), and this can also induce chemical degradation. Manufacturing an MDI formulation as a solution tends, therefore, to lose the prime advantage of the dosage form, which should be to provide a protective, apolar environment, which enhances both chemical and physical stability.

A suspension based MDI overcomes the fundamental flaws associated with solution formulations. A physically stable suspension of a therapeutic agent within a propellant provides a protective environment from which particles can be combined with numerous excipients to potentially achieve a versatile range of drug delivery properties. However, many therapeutic agents require additional stabilising excipients to overcome the problems associated with long-term physical stability within the formulation. The traditional excipients cannot be used for this purpose due to the switch of MDI propellants from CFC's to HFA's.

The formulation and delivery of macromolecules is substantially more difficult than for the more commonly used low molecular weight organic compounds. One of the major reasons for this is added complexity of the structural make up of macromolecules. Proteins, for example, have up to four levels of structural hierarchy including primary, secondary, tertiary and quaternary structures. If such compounds are to be used as therapeutic agents, they must be stored in a formulation and delivered to the site of action with minimal changes to these structural properties, as failure to do so could result in reduction or complete loss of therapeutic activity, and may also lead to immunogenicity.

To date, recombinant human deoxyribonuclease I is the only therapeutic protein specifically formulated for delivery to the lung. Recombinant human deoxyribonuclease is a hydrophilic glycosylated molecule with a molecular weight of ˜33 kDa. It is commercially available as Pulmozymeg in the form of a nebuliser solution. It breaks down the viscosity of lung secretions of cystic fibrosis patients by digesting the endogenous DNA, which can be present at levels of up to 14 mg/ml in some cases. This digestion reduces the viscosity and facilitates the removal of the mucus from the lung (Gonda, 1996). However, atomisation using a nebuliser can deliver less than 30% of the drug to the lungs (Clarke et al., 1993), while the machine is bulky and difficult to use. Further, Pulmozyme® in solution is highly susceptible to heat degradation and has to be stored below 8° C. and hence would not be considered an ideal formulation.

The advantages of delivering proteins using MDI or DPI devices would be significant, if the technological challenges can be overcome. It is of primary importance to maintain the stability of peptide and protein drugs during processing and storage, as well as ensuring the efficiency and reproducibility of the deposition of drug particles during use by the patient. Particular considerations for MDI's include; the production of particles with controlled particle size and stability, and compatibility between propellants and the proteins and peptides. Such factors ensure that the suspension and biological stability can be maintained over the required shelf life.

The stabilisation of proteins using compounds, such as sugars, has enabled these complex macromolecules to be processed using a wide variety of manufacturing techniques with a minimal loss in therapeutic activity (Allison et al., 1999;Aoudia and Zana, 1998a;Aoudia and Zana, 1998b;Byron et al., 1996;Guiavarc'h et al., ;Imamura et al., 2003). Of the numerous processing methods used, spray-drying is one of the most suitable to produce inhalable particles, as the surface morphology of the particle can be manipulated Berggren, 2003; Chan et al., 1997; Harlow, 1993; Prinn et al., 2002; Stahl et al., 2002). However, proteins cannot commonly be spray-dried alone, as the heat used to dry the particles denatures them, so that additional stabilising excipients are required to protect the molecule during the particulate manufacture. Although there have been many previous studies investigating the ability of compounds to protect against the stresses induced during protein manipulation, little has been done to investigate the effects of such excipients on the performance of the final formulation. Although sugars can protect against temperature-induced changes during processing, they do little to protect against solvent-induced protein unfolding, hydrolysis, or aggregation-induced denaturation within a formulation. There is, therefore, a requirement to not only incorporate excipients to protect the protein during manufacture into a suitable particle, but also to maximise stability in the final delivery device.

The compatibility of BFA propellants with protein-powders has been investigated in a number of previous studies. For example, Quinn et al. found that protein MDI formulations retained the biological activity of tested peptides and proteins, such as calcitonin and deoxyribonuclease I, and found that the conformation of lysozyme underwent no change in the presence of HFA-134a as analysed by Fourier transform Raman spectroscopy (Quinn et al., 1999). In other studies, workers from 3M Limited found that protein MDI formulations retained the biological activity of tested peptides and proteins, such as calcitonin and deoxyribonuclease I. Other work also suggests that MDI protein formulations might be efficient in terms of aerodynamic performance and reproducibility, in terms of dosimetry.

Accordingly, if it were possible to provide MDI formulations of protein having both suitable chemical and physical stability during manufacture and storage, then MDI's would have substantial advantages over DPI's for the delivery of appropriate therapeutic substances.

Surprisingly, we have now found that glycosidically stabilised complex drugs, or macromolecules, such as proteins and peptides, have substantially greater stability in the presence of HFA's, when formulated with polyhydroxylated polyalkenes, such as PVA.

Accordingly, in a first aspect, the present invention provides a formulation of a therapeutic substance suitable for delivery to a patient by a metered dose inhalation device, the formulation comprising a substantially dry powder preparation of the substance in association with a stabilising amount of a glycoside and a polyhydroxylated polyalkene in combination with one or more propellants therefor.

In an alternative aspect, the present invention provides a formulation of a therapeutic substance suitable for delivery to a patient by a metered dose inhalation device, the substance being in association with a stabilising amount of a glycoside and being formulated in one or more propellants and/or cosolvent, characterised in that the therapeutic substance is first prepared as a substantially dry powder in the presence of a polyhydroxylated polyalkene, prior to formulation with propellant.

Preferred therapeutic substances are peptides and proteins, and especially those capable of having a therapeutic effect via oral or nasal administration from a metered dose inhaler. The protein or peptide may act in situ, or systemically. A particularly preferred substance is dnase I, preferably human or humanised dnase I, especially dnase I substantially indistinguishable from naturally occurring human dnase I in amino acid sequence or tertiary structure. Human dnase I is most preferred.

In particular, we have now found that dnase, for example, can be formulated with a polyhydroxylated polyalkene and a glycoside in an MDI to retain both biological activity and structural integrity during the production of respirable particles and formulating the particles with BFA propellant. Without these additives, there is a dramatic loss in activity together with structural changes when dnase is spray-dried alone. Thus, it appears that the sugar and the polymer, in combination, protect the protein from both heat-induced denaturation during spray-drying and solvent induced changes upon formulation. It is also an advantage that the formulations of the invention are less likely to be immunogenic, as the additives tend to stabilise the conformation of the active molecule.

Where reference is had to dnase, herein, it will be appreciated that such reference includes reference to all suitable therapeutic substances, unless otherwise apparent, or indicated.

It is a further advantage that the formulations of the invention can be used with portable MDI devices which are easy to use. In addition, the stabilisation of the protein allows it to be stored at room temperature. The delivery efficiency also tends to be higher than with nebulisers, while the delivered protein also generally has significantly greater activity than in a nebulisable formulation.

Therapeutic substances are generally any substances suitable for administration via an MDI device for therapeutic purposes, whether for prophylaxis or treatment. In general, therapeutic substances suitable for use in the formulations of the present invention are advantageously larger, organic molecules, such as peptides and proteins, and may include therapeutic glycosides and steroids, for example. Such molecules may have substantial stability in the presence of BFA's, but the majority of peptides and proteins are not conformationally stable over long periods, and may lose activity, or physical stability, or often both. This loss of activity arises not only through degeneration of the peptide or protein, but also from aggregation of the suspended formulation particles, which serves to reduce the fine particle mass critical for the treatment of the patient.

Such large organic molecules may be stabilised by the presence of suitable glycosidic compounds, particularly the lower oligosaccharides, particularly the di-, tar-, and tetra-saccharides. The terms “glycosides” and “glycosidic compounds” are used interchangeably herein. The composition of the oligosaccharide is not critical to the present invention, and the molecule may comprise a furanosyl residues, pyranosyl residues, straight chain elements, or mixtures thereof. For example, sucrose comprises a furanosyl and a pyranosyl residue, whilst mannitol comprises a pyranosyl residue and a straight chain element. Other suitable disaccharides include lactose, isomaltose, cellobiose, maltose and trehalose, of which trehalose is preferred. Other suitable oligosaccharides include raffinose, melezitose and stachyose. It will be appreciated that the present invention envisages the use of any of these, or other, oligosaccharides either individually or as mixtures. A particularly preferred glycosidic compound is trehalose.

Other glycosidic compounds that may be used include such compounds as mannitol, xylitol, sorbitol, maltitol, isomalt and lactitol. Suitable amounts of the glycosidic compounds are, very approximately, on parity with the therapeutic substance, by weight. More generally, the amount of glycosidic compounds may vary between about 30% and 400% by weight of the therapeutic substance.

It will be appreciated that the glycosidic compounds are preferably simply carbohydrate compounds, but the present invention also includes derivatives thereof, including the glucuronides.

It is an advantage of the present invention that, by combination with a glycoside and a suitably substituted polyhydroxylated polyalkene, the therapeutic substances are now able to be provided in formulations which are stable, even in the presence of haloalkane propellants. It is a particular advantage that such stability is demonstrated in the presence of HFA's, but it will be appreciated that such stability is also demonstrated in the presence of other propellants, such as CFC's, and alkanes, such as butane and propane or combinations of said propellants.

Preferred propellants are the haloalkanes, and it is preferably envisaged that BFA's are used as propellants for MDI's in formulations of the present invention. However, it will be appreciated that the invention also extends to the use of CPC's and other alkanes, for example. The backbone of the propellant will generally be an alkane, whether substituted or unsubstituted, and may be straight or branched. Where branched, it is preferred that there only be one branch. Straight chains of the lower alkanes are preferred, especially C₂₋₄.

The preferred HFA's for use in the present invention are HFA-134a and HFA-227.

Suitable polyhydroxylated polyalkenes for use in the present invention preferably have the structure
—(CH₂—CHOR)ₙ—
where R is the same or different from one monomeric unit to the next, and is hydrogen, lower alkyl, lower alkenyl, lower alkanoyl, lower alenoyl or is a bridging group between adjacent monomers, such as a lower diacyl group. By “lower” is meant 1 to 6 carbon atoms, other than the carbonyl carbon, where present, with 1 to 4 being more preferred, and 1 or 2 being more preferred.

Examples of suitable polyhydroxylated polyalkenes include PVA, PVAc (polyvinylalcohol and polyvinylacetate, respectively),- polyvinyl alcohol-co-vinyl acetate (PVAA), poly(vinyl butyral) and poly(vinyl alcohol-co-ethylene).

PVA is generally prepared by the hydrolysis of PVAc, and the level of hydrolysis may be as low as about 40% through to substantially complete hydrolysis, such as 98% or higher. High levels of hydrolysis correspond to lower levels of hydrophilicity/higher levels of hydrophobicity, which can affect the formulations of the present invention. It is generally preferred that the level of hydrolysis be in the region of 50 to 90%, with a level of about 80% being a preferred embodiment.

The size of the polyhydroxylated polyalkene compounds is not critical to the present invention, and PVA may range from a molecular weight of 9 kDa through to about 500 kDa, with 9 kDa to 50 kDa being more preferred. Where PVA is used as the sole polyhydroxylated polyalkene, then a preferred molecular weight is in the region of 10 kDa. It will be appreciated that molecular weights for the polyhydroxylated polyalkenes are necessarily highly approximate, as the methods for their preparation necessarily result in a spread of molecular sizes.

Suitable amounts of polyhydroxylated polyalkenes range from about 5% to about 200% by weight of the therapeutic substance, although there is little advantage to be seen in the provision of large amounts of the polyhydroxylated polyalene. In general, a suitable amount of polyhydroxylated polyalkene is between about 10% and about 50% by weight of the therapeutic substance with a range of about 20% to about 40% being preferred.

Prior to formulation with the haloalkane propellant, it is preferred to blend the therapeutic agent with the glycosidic compound and polyhydroxylated polyalkene in an aqueous vehicle, prior to drying. The aqueous vehicle may be any suitable, and will typically be selected from saline or a suitable buffer such as phosphate buffered saline (PBS), although deionised water may also be used, if desired.

It will be appreciated that some formulations may comprise two or more populations of particles for administration. In such instances, the glycosides and polyhydroxylated polyalkenes may be selected as appropriate to each substance, and combined with propellant once prepared. It is also possible that, where there are two or more active substances, any two or more may be formulated together.

The powdered products resulting from the drying of the aqueous preparation may be achieved by any suitable drying process, including freeze-drying, spray-drying, spray-freeze-drying, supercritical drying, co-precipitation and air-drying. Of these, spray-drying and spray-freeze-drying are preferred, as these result in fine powders which generally require no further processing. However, if required, the dried products may be further processed to reduce the size of the resulting particles to an appropriate level. In particular, it is preferred that the aerodynamic diameter of the particles of the powder used in the formulations of the present invention is between about 1 μm and 50 μm, more particularly between about 1 μm and 12 μm, and even more particularly between about 1 μm and 10 μm. The dried powder is then brought into contact with the propellants under conditions suitable for storing in a reservoir useful in an MDI.

It is a particular advantage of the present invention that the stability of the particles prepared as described above is considerably greater than anything provided in the art, and preferred formulations of the present invention comprise only the active ingredient(s), glycoside(s), polyhydroxylated polyalkene(s), and propellant(s). Thus, formulations of the present invention provide long-term stability of activity of the therapeutic substance, as well as ensuring consistency of dosing with time.

It will be appreciated that the present invention further provides a powdered formulation of a therapeutic agent, a glycoside and a polyhydroxylated polyalkene suitable for incorporation with a haloalkane propellant for dispensing from a metered dose inhaler.

The present invention further provides a metered dose inhalation device provided with a reservoir comprising a haloalkane propellant prepared with a therapeutic substance, a glycoside and a polyhydroxylated polyalkene.

Doses delivered by the MDrs of the present invention will be readily determined by those skilled in the art and as appropriate to the condition to be treated. In general, doses will vary with the size and age of the patient and can be readily determined by calculating the concentration of the active ingredient in the propellant preparation.

Suitable macromolecular compounds for use as therapeutic agents include antibodies, interferon, such as α-interferon, β-interferon and γ-interferon, enzymes such as proteases and ribonucleases, especially DNase L hormones, such as insulin, LHRH, granulocyte-colony stimulating factor, calcitonin, heparin, human growth hormone, euprolide acetate and parathyroid hormone and gene products such as CFTR, and α1-antitrypsin. Other large and/or complex molecules or structures may also be incorporated in MDI formulations, in accordance with the present invention.

The present invention will now be further illustrated by the following, non-limiting examples.

### EXAMPLE 1

Compositions and Spray-Drying Process for Preparing Particles

Particles suitable for admixture with a propellant mixture were prepared as follows. Buffer phosphate salts (ACS reagent grade), sodium chloride, PVA (MW, 9,000-10,000), sucrose, trehalose, lysozyme, and catalase were purchased from Sigma-Aldrich Co.

Enzymes and excipients were dissolved in buffer or saline and spray-dried using a Model 190 Butchi mini spray-dryer. The solutions employed to dissolve lysozyme and catalase were 5 mM sodium phosphate buffer (pH 6.2) and 5 mM potassium phosphate buffer (H 7.0), respectively, and the enzyme concentrations were maintained at 5 mg/ml. The compositions of the spray-dried formulations are shown in Table 1, below.

TABLE 1
The compositions and designations of spray-dried enzyme formulations.

| Formulation | Composition |
|---|---|
| LO1:0 | Lysozyme 5 mg/ml |
| LS1:1 | Lysozyme 5 mg/ml + sucrose 5 mg/ml |
| LT1:1 | Lysozyme 5 mg/ml + trehalose 5 mg/ml |
| LPT5:0.5:5.5 | Lysozyme 5 mg/ml + PVA 0.5 mg/ml + trehalose |
| | 5.5 mg/ml |
| LPT5:1:6 | Lysozyme 5 mg/ml + PVA 1 mg/ml + trehalose 6 mg/ml |
| LPT5:2:7 | Lysozyme 5 mg/ml + PVA 2 mg/ml + trehalose 7 mg/ml |
| LPT1:1:2 | Lysozyme 5 mg/ml + PVA 5 mg/ml + trehalose 10 mg/ml |
| CO1:0 | Catalase 5 mg/ml |
| CS1:1 | Catalase 5 mg/ml + sucrose 5 mg/ml |
| CT1:1 | Catalase 5 mg/ml + trehalose 5 mg/ml |
| CPT5:1:6 | Catalase 5 mg/ml + PVA 1 mg/ml + trehalose 6 mg/ml |

The feed solution was pumped peristaltically through a silicone tube (3 mm) to a two fluid nozzle (0.5 mm) head used to atomise the fluid. Cooling water (0° C.) was circulated through the jacket around the nozzle at a-rate of about 36 ml/min. The processing parameters were: a feed rate of 3 ml/min; an atomising air-flow rate of 700 l/h; and an inlet temperature of 95° C. Outlet temperatures were found to range from 65 to 69° C. The solution volume employed to produce each spray-drying batch was 100 ml and each process lasted 34 min. The protein powders were collected in a collection jar, after all the feed solution had been processed, but without allowing time for the powder to cool to room temperature, the material was transferred to a 7 ml vial, which was immediately sealed by capping. This vial was then transferred to a freezer (−20° C.) for storage.

### EXAMPLE 2

Relative Enzyme Activity Remaining After Spray-Drying

The activity of the enzyme in each formulation is shown in Table 2. Spray-dried lysozyme was found to retain about 87% of the original activity, whilst those formulations containing excipients appeared to maintain almost the full activity of the original enzyme. Inactivation of catalase upon spray-drying was found to be about 55% of the initial activity, but the loss of activity was reduced to about 7% when either sucrose or trehalose was included, and almost full activity was recovered when a PVA-trehalose mixture was included in the formulation.

TABLE 2
Recovered biological activity in spray-dried lysozyme and
catalase particles (Mean ± SD, n = 3).

| Formulation | Relative activity (%) | Formulation | Relative activity (%) |
|---|---|---|---|
| LO1:0 | 87.2 ± 2.1 | LPT1:1:2 | 95.9 ± 3.0 |
| LS1:1 | 97.2 ± 3.0 | CO1:0 | 54.4 ± 4.1 |
| LT1:1 | 96.9 ± 3.2 | CS1:1 | 92.3 ± 2.8 |
| LPT5:0.5:5.5 | 100.4 ± 2.2 | CT1:1 | 93.38 ± 2.1 |
| LPT5:1:6 | 97.1 ± 3.7 | CPT5:1:6 | 99.8 ± 2.8 |
| LPT5:2:7 | 97.3 ± 4.8 | | |

### EXAMPLE3

Geometric Particle Size of Spray-Dried Particles

The particle size as well as size distribution of the spray-dried protein particles are shown in Table 3. The volume median diameters (VMD) of all spray-dried particles were found to be between 2.48 and 3.43 μm. In addition, the span of particle size distribution was found to be between 0.77 and 1.18, which indicates that all the powders exhibited a relatively high degree of monodispersity, whilst the upper limit of the size range of the particles appeared to be ≦12.5 μm.

TABLE 3
Particle size and distribution of spray-dried lysozyme
and catalase formulations.

| | Median diameter | | Size range |
|---|---|---|---|
| Formulation | (μm) | Span | (μm) |
| LO1:0 | 3.09 | 0.77 | 1.22-7.49 |
| LS1:1 | 3.43 | 0.92 | 1.22-12.5 |
| LT1:1 | 3.31 | 0.91 | 1.22-11.6 |
| LPT5:0.5:5.5 | 2.48 | 1.15 | 1.22-7.49 |
| LPT5:1:6 | 2.67 | 1.03 | 1.22-9.31 |
| LPT5:2:7 | 2.78 | 1.18 | 1.22-10.0 |
| LPT1:1:2 | 2.89 | 1.13 | 1.22-10.8 |
| CO1:0 | 3.12 | 1.18 | 1.22-10.0 |
| CS1:1 | 2.95 | 1.04 | 1.22-9.31 |
| CT1:1 | 2.96 | 1.15 | 1.22-10.0 |
| CPT5:1:6 | 2.77 | 1.33 | 1.22-10.0 |

### EXAMPLE 4

The Effect of HFA on the Biological Activity of Spray-Dried Enzymes

For spray-dried lysozyme and catalase in the presence of sucrose, trehalose or a trehalose-PVA mixture, there appeared to be no detectable reduction in activity after being stored in a BFA based-MDI canister up to 26 weeks, whilst the activity of spray-dried catalase alone was found to be reduced to ˜20% within 12 weeks (Table 4).

TABLE 4
The retained activity of HFA based MDI-formulated lysozyme and
catalase particles relative to the corresponding control powders
after storage for 12 weeks at room temperature.

| | | Activity of MDI- |
|---|---|---|
| | Activity of MDI-formulated | formulated enzyme at |
| Formulation | enzyme at Week-1 (%) | Week-12 (%) |
| LO1:0 | 99.0 | 98.1 |
| LPT5:0.5:5.5 | 96.8 | 100.0 |
| LPT5:1:6 | 102.9 | 95.3 |
| LPT5:2:7 | 98.9 | 99.0 |
| LPT1:1:2 | 97.6 | 93.0 |
| CO1:0 | 98.9 ± 2.7 | 20.8 ± 6.8 |
| CPT5:1:6 | 101.1 ± 4.7 | 98.2 ± 1.8 |

### EXAMPLE5

Deposition of MDI-Formulated Lysozyme Particles

The in vitro deposition performance of MDI-formulated spray-dried lysozyme particles is shown in Table 5. For the formulation prepared using spray-dried lysozyme alone, the protein fractions recovered from the device, stage 1 and stage 2 were found to be about 14.6, 34.9 and 50.5% respectively, during the first week after preparation. After storage at room temperature for up to 12 weeks, the stage 2 fraction significantly decreased to 42.7% whilst the stage 1 fraction increased to 42% of the recovered dose (p<0.05, one tailed student t-test, Table 5).

When lysozyme was stabilised using either sucrose or trehalose as excipient during spray-drying, the aerodynamic properties of the resultant MDI formulations were significantly affected (p<0.05, two tailed student t-test). At the first week after manufacture, the stage 2 fraction of MDI formulation LS 1:1 appeared to decrease to 27.2% whilst the fraction recovered from the device and stage 1 increased to 21.7 and 51.3% respectively. After 6 weeks storage at room temperature, the stage 2 fraction decreased significantly to about 8% (p<0.05, two tailed student t-test). However, with further storage for up to 26 weeks, there appeared to be no more reduction in the stage 2 fraction. The MDI formulated LT1:1 particles displayed a similar aerodynamic performance to the LS1:1 formulations at the first week after preparation. However, the storage suspension stability of the former proved to be significantly better than the latter (p<0.05, paired student t-test). Nonetheless, the fine particle fraction (stage 2 fraction) of LT1:1 MDI formulation was susceptible to decrease as a function of storage time. After stored for 26 weeks, the fine particle fraction significantly decreased to 12.7% (p<0.05, two tailed student t-test) whilst the stage 1 fraction increased to 61.4% of the recovered protein.

When lysozyme was spray-dried in the presence of PVA-trehalose mixture, the resultant MDI formulations appeared to have significantly better aerosol performance than those formulated using either trehalose or sucrose alone in combination with the enzyme (p<0.05, two tailed student t-test, Table 5). The fine particle fraction of MDI formulations containing PVA were found to range from 47.1 to 52.7% at the first week after preparation. The stability of aerodynamic properties was found to depend upon the PVA content in the spray-dried particles. After storage for 12 weeks at room temperature, the formulation (LPT5:0.5:5.5) containing the lowest PVA content was found to emit a insignificantly decreased fine particle fraction of 42.8%, in comparison to the 48.3% obtained during week-1 (P>0.05, one tailed student t-test). The other formulations containing a higher ratio of PVA content in the formulations appeared to retain a constant fine particle fraction over the 12 week storage period. All the PVA containing MDI formulations displayed a significantly better storage suspension stability, in terms of fine particle fraction, than either the MDI LS1:1 or LT1:1 formulations p<0.05, paired student t-test).

TABLE 5
The aerosol performance of HFA based MDI lysozyme formulations
as evaluated by a twin stage impinger after storage up to
12 weeks (Mean ± SD, n = 3).

| | | Fraction at | Fraction at week- | Fraction at |
|---|---|---|---|---|
| Formulation | Stage | week-1 (%) | 6 (%) | week-12 (%) |
| LO1:0 | Device | 14.59 ± 3.21 | 15.41 ± 2.55 | 15.29 ± 3.11 |
| | Stage 1 | 34.90 ± 2.75 | 34.27 ± 1.85 | 42.01 ± 2.89 |
| | Stage 2 | 50.51 ± 3.76 | 50.32 ± 0.81 | 42.69 ± 3.41 |
| LPT5:0.5:5.5 | Device | 15.97 ± 2.9 | 10.92 ± 1.05 | 15.12 ± 3.56 |
| | Stage 1 | 37.54 ± 3.14 | 40.88 ± 2.73 | 42.07 ± 2.51 |
| | Stage 2 | 48.27 ± 3.3 | 48.20 ± 3.65 | 42.80 ± 3.89 |
| LPT5:1:6 | Device | 15.20 ± 1.10 | 15.30 ± 2.06 | 16.41 ± 2.78 |
| | Stage 1 | 39.33 ± 6.08 | 39.39 ± 1.97 | 38.72 ± 4.10 |
| | Stage 2 | 47.14 ± 9.12 | 45.30 ± 0.30 | 44.87 ± 3.67 |
| LPT5:2:7 | Device | 18.29 ± 2.23 | 10.87 ± 1.03 | 13.49 ± 1.32 |
| | Stage 1 | 33.18 ± 6.60 | 41.65 ± 1.76 | 37.92 ± 2.87 |
| | Stage 2 | 48.54 ± 4.46 | 49.11 ± 3.25 | 48.59 ± 3.2 |
| LPT1:1:2 | Device | 19.32 ± 2.97 | 13.35 ± 1.70 | 12.47 ± 3.21 |
| | Stage 1 | 30.57 ± 1.49 | 33.30 ± 4.38 | 32.10 ± 2.98 |
| | Stage 2 | 52.73 ± 1.97 | 54.42 ± 3.10 | 55.44 ± 3.70 |

### EXAMPLE 6

Deposition of MI-Formulated Catalase Particles

The in vitro deposition performance of MDI catalase formulations is shown in Table 6. For the MDI-formulated spray-dried catalase without any excipient, the protein fractions recovered form device, stage 1 and stage 2 were found to be about 23.7, 43.3 and 33.0% respectively, during the first week after preparation. However, after the same formulations had been stored for 6 weeks at room temperature, the stage 2 fraction was found to decrease drastically to almost 0% with about 89% of particles being deposited in stage 1 (Table 6).

TABLE 6
The aerosol performance of HFA based MDI-formulated catalase particles
as evaluated by a twin stage impinger after storage up to 12 weeks.

| | | Fraction at | Fraction at week- | Fraction at |
|---|---|---|---|---|
| Formulation | Stage | week-1 (%) | 6 (%) | week-12 (%) |
| CO1:0 | Device | 23.71 ± 2.09 | 10.00 ± 1.02 | ND |
| | Stage 1 | 43.31 ± 1.32 | 88.79 ± 1.29 | ND |
| | Stage 2 | 32.98 ± 0.90 | 1.20 ± 2.07 | ND |
| CPT5:1:6 | Device | 15.84 ± 3.01 | 13.16 ± 2.58 | 17.85 ± 1.31 |
| | Stage 1 | 25.24 ± 1.00 | 32.47 ± 1.46 | 30.74 ± 4.77 |
| | Stage 2 | 58.92 ± 3.02 | 54.37 ± 3.89 | 53.26 ± 6.08 |

The spray-dried catalase formulation containing either sucrose or trehalose as stabiliser, produced a significantly higher stage 2 deposition of protein relative to the MDI CO1:0 formulation, as evaluated during the first week after manufacture (p<0.05, two tailed student t-test). The stage 2 fractions of MDI formulated CS1:1 and CT1:1 appeared to increase from 33.0% in the absence of excipient to 39.3 and 44.8% respectively, when sucrose or trehalose were employed. The stage 1 fractions appeared to be almost identical in the absence or presence of excipient. The fine particle fractions generated by the CS1:1 and CT1:1 MDI formulations appeared to decrease as a function of storage time. The formulation incorporating trehalose emitted a higher fine particle fraction after 6-26 weeks of storage than the similar formulation containing sucrose. For example, after 26 weeks storage at room temperature, the stage 2 fraction of the CS1:1 MDI formulation was 6.0%, relative to the 18.7% emitted from MDI containing the CT1:1 formulation. The reductions in the fine particle fractions were compensated by increases in the stage 1 fractions, whilst the device fractions were consistently found to be about 20% of the recovered dose and independent of formulation and storage time.

The fine particle fraction of the PVA containing MDI formulation was found to be 58.9%, which was significantly higher than that of the MDI formulated CS1:1 or CT1:1 particles (p<0.05, two tailed student t-test), whilst the device and stage 1 fractions accounted for only 15.9 and 25.2% of the recovered does respectively, as evaluated during the first week after preparation. After storage for 6 weeks at room temperature, a slight decrease in fine particle fraction was found, albeit not significant (p>0.05, one tailed student's t-test). Moreover, after storage for a further 6 weeks, the recovered fine particle fraction appeared to be the same. When catalase was spray-dried in the presence of PVA-trehalose mixture, the resultant MDI formulations appeared always to display a significantly better aerosol performance in comparison to the MDI formulated CS1:1 or CT1:1 particles during storage (p<0.05, paired student t-test).

### EXAMPLE 7

Stabilisation of a Dnase I Metered Dose Formulation

While the human form of deoxyribonuclease (dnase) is used for clinical applications, its manufacture and purification is costly. However, the bovine form of the protein provides an excellent model. The sequences of the human and bovine forms are 77% homologous and the crystal structures can be superimposed upon each other (Quan et al., 1999). In the following Example, highly purified bovine deoxyribonuclease I was reformulated in a metered dose inhaler preparation, and the ability of trehalose and polyvinyl alcohol to stabilise bovine dnase I during manufacture using spray-drying and formulation in a metered dose inhaler was assessed, by comparison with spray-drying the raw enzyme alone.

Deoxyribonuclease I (isolated from the bovine pancreas, high purity, Rnase free, 14200 U/mg (defined by Sigma Aldrich as Genotech® units) Sigma Aldrich, Gillingham, UK) formulations were manufactured using the Bucchi 191 mini spray-dryer (B3ucchi, Darmstadt, Germany). The aspiration rate was set as 70%, the material feed rate was 3 ml min⁻¹ and the inlet temperature was set to 95° C. The feed suspension was pumped through a spray atomisation nozzle that combined the liquid with a 700 ml hr⁻¹ airflow. The outlet temperature was determined by the previously detailed parameters but was consistently found to be in the range 65-70° C.

The dnase spray-drying feed solutions were made up in 100 ml of 0.15M NaCl buffer. Two formulations were manufactured in total as detailed in Table 7, below. The PVA was 80% hydrolysed with a molecular weight (M,) of 8,000-10,000 (Sigma Aldrich, Gillingham, UK). The trehalose was in the dihydrate form (Sigma Aldrich, Gillingham, UK).

TABLE 7
Composition of the Deoxyribonuclease I spray-dried formulations

| Formulation | Composition |
|---|---|
| DO1:0 | dnase I 5 mg/ml |
| DTPVA 1:1:1 | dnase I 5 mg/ml + trehalose 5 mg/ml + PVA 80% |
| | hydrolysed 5 mg/ml |

The product from the spray-drying process was collected and weighed into a glass vial. The samples were stored under phosphorous pentoxide desiccation at room temperature for 24 hours prior to MDI manufacture.

TABLE 8
Composition of the Deoxyribonuclease I MDI formulations

| Formulation | Composition |
|---|---|
| DO1:0 134a | DO1:0 15.0 mg + HFA 134a 15.0 g |
| DTPVA 1:1:1 | DTPVA 1:1:1 45.0 mg + HFA 134a 15.0 g |
| 134 a | |
| DTPVA 1:1:1 | DTPVA 1:1:1 45.0 mg + HFA 227 17.0 g |
| 227 | |

The metered dose inhalers were manufactured by adding the equivalent of 15.0 mg of the raw drug (dnase) into a PET canister (BesPack, Kings Lynn, UK), so that 15.0 mg of DO1:0 and 45.0 mg of DTPVA 1:1:1 were used. A total of three formulations were manufactured, as detailed in Table 8, above. A 25 μL canister valve (BesPack, Kings Lynn, UK) was crimped in place using the Pamasol MDI filler (Pamasol, Pfaffikon, Switzerland) and 15.0 g of HFA 134a (Dupont, Willington, Germany) or 17.0 g HFA 227 (Solvay, Frankfurt, Germany) was pressure-filled into the can via the valve. The formulation was then sonicated in an ultrasonication bath (Decon, Hove, UK) for 15 seconds to ensure particle separation and stored, valve up, at room temperature. The denatured dnase used as a positive control was simply manufactured by placing 5.0 mg of the protein in a 180° C. oven for 10 minutes.

Particle Size Analysis

The spray-dried powders were assessed using the Mastersizer X laser diffraction particle size analyser (Malvem Instruments Ltd, Malvem, UK). The Malvem was set up using the liquid dispersion system. Mixtures of 1% lecithin (Sigma Aldrich, Gillingham, UK) and cyclohexane (Merck, Poole, UK) were used as the dispersion media. Samples were prepared by sonicating 2 mg of powder in 2 ml of the dispersion media for 30 seconds. The particle size was measured using the 63 mm (0.5-110 μm) lens set at a focal length of 145 mm, whilst stirring the cell on 75% of full power. The samples were added dropwise in to the stirred cell until the desired obscuration was achieved. Each sample was measured in triplicate and 3 batches from each sample were analysed.

Biological Activity

The biological activity of dnase I was monitored by assessing the enzyme's ability to digest the substrate, DNA. The substrate was made up in an acetate buffer (0.1 M, pH 5.0), containing 5 MM Mg²+. This was prepared by dissolving 1.165 g of anhydrous sodium acetate (BDH, Merck labs, Darmstadt, Germany), 0.355 g of acetic acid (Sigma Aldrich, Gillingham, UK), and 0.203 g of MgCl₂.6H₂O (Sigma Aldrich, Gillingham, UK), in 150 ml of purified water. 2 mg of fibrous DNA isolated from a calf thymus (Sigma Aldrich, Gillingham, UK) was dissolved in 52 ml of the acetate buffer by gently shaking overnight. The absorbance of this substrate solution at 260 nm was determined to be between 0.630 and 0.690.

Prior to assessing the test samples, a dnase I standard, 2,000 Kunitz units mg⁻¹ (Sigma Aldrich, Gillingham, UK), was used as a calibrant for the activity assay. This standard was reconstituted by dissolving it in 1.0 ml of 0.15 M NaCl solution. The solution was further diluted to obtain five separate standard solutions within the concentration range of 20-80 units ml⁻¹. All dilutions were performed using 0.15 M NaCl solution.

A lambda 5 UV spectrophotometer (Perkin-Elmer, Beaconsfield, UK) was adjusted to a wavelength of 260 nm and 2.5 ml of substrate was placed into a cuvette (10 mm light path) and incubated in a thermostatic cell (25° C.) for 3-4 minutes to allow temperature equilibration. Then, 0.5 ml of diluted standard, or sample, was added and the solutions were immediately mixed by inversion. The increase in A₂₆₀ (ΔA₂₆₀) minutes was recorded as a function of time for 10-12 minutes. An activity calibration curve was constructed by plotting the maximum ΔA₂₆₀ vs. Kunitz units mg⁻¹ of the standard dnase I vials. The dnase samples were diluted to attain a ΔA₂₆₀ within the calibration range and, hence, measure the equivalent Kunitz units. The Pierce Protein Assay® was then used to quantify the protein, thereby to obtain the activity per mg. This was compared to the lyophilised raw dnase I to produce the % relative activity.

Twin-Stage Impinger

The twin stage impinger (Radleys, Saffron, UK) was set up as per the United States Pharmacopoeia specification. The dnase I formulations used distilled water as washing agent and the solvent in the apparatus. The airflow was set to 60 ml min⁻¹ and the inhalers were actuated 20 times. Between each actuation there was a five second pause with the pump running. The pump was then stopped, the canister removed and shaken for five seconds before the sequence repeated. Each of the stages were washed individually upon completion of the 20 canister actuations. The device was washed into a 50 ml volumetric with stages 1 and 2 being washed into 100 ml volumetric flasks. The resulting solutions were analysed using the Pierce Protein Assay® (Pierce Chemical Company, UK). All twin stage runs were completed in triplicate.

The Pierce Protein Assay® was performed as per the manufacturer's instructions. BSA was used as the protein standard and a set of BSA solutions between 2 and 20 μg were prepared by diluting the 2.0 mg ml⁻¹ standard. The working reagent was prepared by mixing 25 parts of Micro BCA reagent A and 24 parts of reagent B with 1 part of reagent C. An aliquot of 150 μL of each standard or test sample was transferred into a 96-well microplate in duplicate. 150 μL of the working reagent was subsequently added to each well and the plate mixed on the shaker for 30 seconds. The plate was covered and incubated at 50° C. for 90 minutes, after which it was cooled to room temperature and the UV absorbance in each well determined at 562 nm using a UV plate reader. The response of each enzyme was determined by comparing the nominal concentration and the BSA protein standard.

Fluorescence

Fluorescence emission and Rayleigh light scattering were both assessed using a LS-50 fluorescence spectrophotometer with a thermostatic cell set at 5° C. (Perkin-Elmer, Beaconsfield, UK). The excitation wavelength was set to 270 nm and the emission was monitored over a range of 250 nm to 450 nm. The excitation slit width was set as 4 nm and the emission slit width 8 mn. The spectra were attained at a rate of 150 nm. All the samples were made up in a 0.15 M NaCl solution (Sigma Aldrich, Gillingham, UK). The samples were each scanned five times and averaged. The spectra from the solvent were subtracted from each result. The area under the light scattering peak (maximum cc. 270 nm) and the fluorescence peak (maximum cc. 335 nm) were integrated from each sample and compared. The light source variance was assessed and, if appropriate, corrected for, using Nile Red (Sigma Aldrich, Gillingham, UK) as a standard.

Spray-Dried Material Characterisation

The two formulations were manufactured using the Bucchi spray-dryer. The particle size measurements of the spray-dried material indicated that both of the batches were of a suitable respirable size, i.e. less than 10 μm. The results are shown in Table 9. The smallest mean particle size (2.25±0.05 μm) was produced by simply spray-drying the protein alone.

TABLE 9
Dnase I spray-dryer manufacture yield and particle size distribution
(Mean ± SD, n = 3).

| | D[v, 0.1] | D[v, 0.5] | D[v, 0.9] | |
|---|---|---|---|---|
| Formulation | μm | μm | μm | % Yield |
| DO1:0 | 1.00 ± 0.02 | 2.25 ± 0.05 | 4.52 ± 0.52 | 15.40 |
| DTPVA 1:1:1 | 1.24 ± 0.01 | 3.06 ± 0.12 | 6.57 ± 0.43 | 34.75 |

The yields for the operation were improved with the addition of the excipients. Incorporation of trehalose and PVA more than doubled the yield of the manufacture method from 15.40% to 34.75%.

The Effect of HFA on the Dnase I Formulations

The biological activity of the enzyme, dnase I, was reduced by almost 40% when spray-dried alone from a 0.15 M NaCl solution. However, over 85% of the biological activity was retained when PVA and trehalose were incorporated into the feed solution. Results are shown in column 2 of Table 10, below. The additional excipients showed a significant improvement (p<0.05) in the retention of the enzyme's activity compared to the enzyme spray-dried alone.

TABLE 10
Relative biological activity of the spray-dried dnase particles before
and after formulation in HFA propellants (Mean ± SD, n = 3).

| Dnase Particles | | HFA Formulation | |
|---|---|---|---|
| | Relative | | Relative |
| Formulation | activity (%) | Formulation | activity (%) |
| DO1:0 | 63.86% ± 4.23 | DO1:0 134a | 61.43% ± 3.88 |
| DTPVA 1:1:1 | 85.34% ± 2.18 | DTPVA 1:1:1 134a | 95.91% ± 10.98 |
| | | DTPVA 1:1:1 227 | 103.06% ± 4.50 |

Formulation of the DO1:1dnase I particles with HFA 134a had no significant effect on the activity of the enzyme, as shown in column 4 of Table 10. However, surprisingly, addition of the DTPVA 1:1:1 particles to both BFA 134a and HFA 227 reversed the 15% lost activity to return the enzyme to its original potency. There was no significant difference between the activity of the dnase (p<0.05) in either of the HFA propellants. These results suggest that the when the protein is spray-dried alone, denaturation is occurring, but that this is prevented by combination with the excipients. The addition of PVA and trehalose results in total retention of the protein's activity, suggesting that, within the DTPVA 1:1:1 formulation, the active site of the protein, i. e. the area of the protein that digests the DNA, has remained intact.

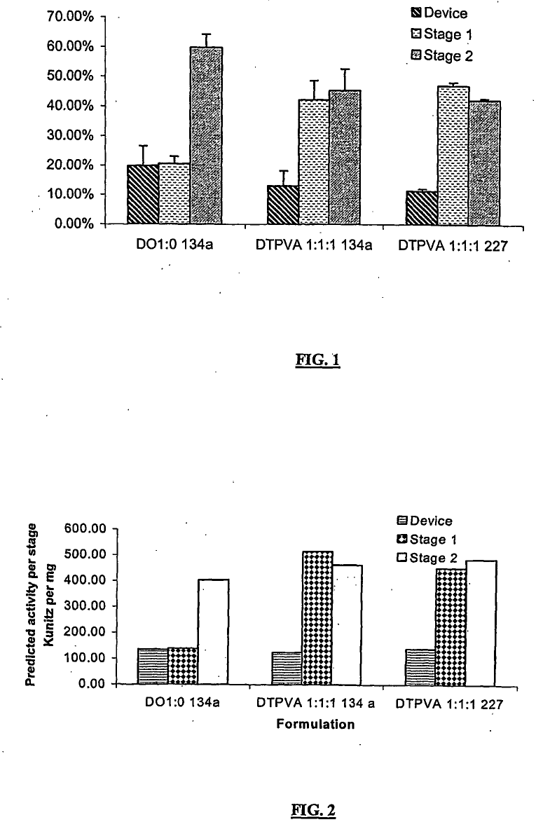

In vitro prediction of particle deposition using the twin stage impinger apparatus defines the fine particle fraction as the particles collected on stage 2 of the device. Stage 2 has a size cut off MMAD of <6.4 μm. In accompanying FIG. 1, there is presented the impaction data for the three dnase MDI formulations, determined in vitro using a twin stage impinger. Formulations are detailed in Table 9.

Both sets of the spray-dried dnase particles (DO1:0 and the DTPVA 1:1:1) produced a high FPF in the twin stage impinger apparatus when suspended in HFA propellants. DO1:0 134a delivered a significantly higher (p<0.05) FPF compared to either the DTPVA 1:1:1 134a or the DTPVA 1:1:1 227 formulation (which were not significantly different (p<0.05) from each other), see FIG. 1. However, the twin stage impinger data should not be analysed in isolation. Both the percentage of dnase delivered to the second stage of the device and the activity of the particles delivered must be taken into account to attain a true prediction of formulation efficiency (FIG. 2). The accompanying FIG. 2 shows the combination of enzyme activity data and twin stage impinger data to predict the quantity of active enzyme delivered to the lung.

Although the DO1:0 formulation delivers a high proportion of particles to stage two, only 60% of these retained biological activity. The DTPVA 1:1:1 formulation, on the other hand, retained fall biological activity and, therefore, when suspended in HFA, delivered more active particles to stage 2. There was no difference between the two types of HFA propellants in this case, hence DTPVA 1:1:1 suspended in either HFA 134a or HFA 227 is the most efficient delivery vehicle for the protein. Both formulations deposited almost 50% of the actuated dose on stage 2 of the TSI device.

Rayleigh light scattering is measured at 90 degrees to the incident light. The Rayleigh emission from particulates within solutions occurs at the same wavelength at which it was applied to a sample. As more aggregates are formed within a solution, the intensity of the Rayleigh light scattering increases. Therefore, measurement of Rayleigh light emission has been previously used to monitor the aggregation of protein solutions. Aggregation follows secondary structure breakdown in a protein and, therefore, may be indicative of protein denaturation. Furthermore, the tryptophan residue in a protein is known to be fluorescent. Although this is not a unique property of amino acids (both tyrosine and phenylalanine also fluoresce) the fluorescence of the tryptophan residue is uniquely sensitive to its microenvironment. Structural changes in a protein, such as unfolding or aggregation, can lead to change in the microenvironment of the tryptophan residue, which results in a change in fluorescent intensity, due to quenching or intensity maxima, through a variation in hydrophobicity of the microenvironment. Hence, monitoring of Rayleigh light scattering (which can be performed in a single scan on a fluorescence spectrophotometer) and fluorescence can both indicate structural changes on both a macro and-Micro-environmental level.

TABLE 11
Integrated light scattering and fluorescence peaks for the dnase I
spray-dried material.

| | Raleigh Peak | Fluorescence Peak | Fluorescence |
|---|---|---|---|
| Formulation | Area | Area | Maximum |
| DO1:0 | 73529 ± 4898 | 1546667 ± 202072 | 334.5 ± 0.5 |
| DTPVA 1:1:1 | 1241 ± 2149 | 916143 ± 18726 | 336.5 ± 0.5 |
| DO1:0 134a | 74036 ± 1530 | 1165807 ± 24109 | 333 ± 0.5 |
| DTPVA 1:1:1 134 a | 24516 ± 247 | 684487 ± 7210 | 337 ± 0 |
| DTPVA 1:1:1 227 | 17647 ± 353 | 903542 ± 18070 | 336 ± 0 |

Assessing the particles prior to suspension in HFA propellant highlighted the fact that the DTPVA 1:1:1 spray-dried formulation produced a significantly lower (p<0.05) Rayleigh light scattering peak area, compared to the DO1:0 particles, implying less protein aggregates were present (see Table 11, above). However, the spray-dried dnase, DO1:0, produced a significantly (p<0.05) larger peak area for the fluorescence emission of tryptophan, compared to the DTPVA 1:1:1 spray-dried formulation. Although the fluorescence maxima were significantly different (p<0.05) for the two dnase batches, the differences were so small that they were not considered indicative of changes in the protein structure. Upon suspension of the dnase particles in HFA, changes were observed with both the Rayleigh light scattering and the fluorescence emission spectra (again, the small shifts in peak maxima, although statistically significant, were not considered, due to the fact the shifts were so small they were thought not to be indicative of structural changes). DO1:0 134 showed no significant change in Rayleigh light scattering. However, it did show a significant drop (p<0.05) in fluorescence emission from a peak area of 1546667 to a peak area of 1165807. DTPVA 1:1:1 134a also showed a drop in fluorescence intensity after formulation in propellant, this was coupled with a significant rise p<0.05) in Rayleigh light scattering. DTPVA 1:1:1 227 observed a similar increase in Rayleigh light scattering to DTPVA 1:1:1 134a, however, the fluorescence intensity remained constant both before and after incorporation with BFA propellant.

The changes in both Rayleigh light scattering and fluorescence intensity infer that both the DO1:0 and the DTPVA 1:1:1 spray-dried particles change upon suspension in BFA propellant. Only minimal changes occurred with the DTPVA 1:1:1 227 formulation, indicative of enhanced stability for the formulation.

Compared to a simple spraydried dnase formulation, incorporation of the trehalose and PVA with the protein increased the yield of the manufacture method, improved the retention of the protein's activity, both before and after suspension in HFA, and maximised the secondary structure integrity throughout. The PVA/ trehalose formulation also minimised aggregate formation and slowed or prevented changes in the microenvironment of the tryptophan residue.

The protection of the protein during both manufacture and the formulation of dnase I, using trehalose and PVA as excipients, produced a MDI formulation that was both aerodynamically suitable for lung delivery and therapeutically active. The formulations held the protein within its native state from the point of manufacture to its delivery from the MDI device, which maximised its stability and minimises any potential immunological responses by the body when it is delivered in vivo.

### REFERENCES CITED

Allison,S D, B Chang, T W Randolph, J F Carpenter, 1999, Hydrogen bonding between sugar and protein is responsible for inhibition of dehydration-induced protein unfolding: Archives of Biochemistry and Biophysics, v. 365, p. 289-298.
Aoudia,M, R Zana, 1998a, Aggregation behavior of sugar surfactants in aqueous solutions: Effects of temperature and the addition of nonionic polymers: Journal of Colloid and Interface Science, v. 206, p. 158-167.
Aoudia,M, R Zana, 1998b, Aggregation Behavior of Sugar Surfactants in Aqueous Solutions: Effects of Temperature and the Addition of Nonionic Polymers: Journal of Colloid and Interface Science, v. 206, p. 158-167.
Berggren,J A G, 2003, Effect of polymer content and molecular weight on the morphology and eat- and moisture-induced transformations of spray dried composite particles of amorphous lactose and poly(vinylpyrrolidone): Pharmaceutical Research, v. 20, p.1039-1046.
Blondino,F E, P R Byron. Drug stability in non-aqeous solutions-influence of siirfactant concentration. V, 125-131. 1998. Respiratory Drug Delivery. Ref Type: Conference Proceeding
Byron,PR, V Naini, E M Phillips. Drug carrier selection-important physicochemical characteristics. V, 103-113. 1996. Respiratory Drug Delivery. Ref Type: Conference Proceeding
Chan,H K, A Clark, I Gonda, M Mumenthaler, C Hsu, 1997, Spray dried powders and powder blends of recombinant human deoxyribonuclease (rhDNase) for aerosol delivery: Pharmaceutical Research, v. 14, p. 431-437.
Clarke,J G, S R Wicks, S J Farr, 1993, Surfactant-Mediated Effects in Pressurized Metered-Dose Inhalers Formulated As Suspensions .1. Drug Surfactant Interactions in A Model Propellant System: International Journal of Pharmaceutics, v. 93, p. 221-231.
Gonda,I, 1996, Inhalation therapy with recombinant human deoxyribonuclease I: Advanced Drug Delivery Reviews, v. 19, p. 37-46.
Guiavarc'h,Y, D Sila, T Duvetter, A Van Loey, M Hendrickx, kIfluence of sugars and polyols on the thermal stability of purified tomato and cucumber pectinmethylesterases: a basis for TTI development: Enzyme and Microbial Technology, v. In Press, Corrected Proof.
Harlow, 1993, Spray drying handbook, UK longman scientific and technical. Hochhaus,G, S Suarez, R J Gonzalez-Rothi, H Schreier. Pulmonary targeting of inhaled glucocorticoids: How is it influenced by formulation. VI, 45-52. 1998. Respirtory Drug Delivery. Ref Type: Conference Proceeding
Imamura,K, T Ogawa, T Sakiyama, K Nakanishi, 2003, Effects of types of sugar on the stabilization of protein in the dried state: Journal of Pharmaceutical Sciences, v. 92, p. 266-274.
Koppenhagen, I, et al (2001), Evaluation of pressurised metered dose inhalers for pulmonary delivery of proteins and peptides, www.3m.com/us/healthcare/manufacturers/dds/pdf/pub_{—}2001_{—}09_koppenhagen.pdf
Leach,C L, P J Davidson, B E Hasselquist, R J Boudreau, 2002, Lung deposition of hydrofluoroalkane-134a beclomethasone is greater than that of chlorofluorocarbon fluticasone and chlorofluorocarbon beclomethasone—A cross-over study in healthy volunteers: Chest, v. 122, p. 510-516.
LeBelle,M, R K Pike, S I Graham, E D Ormsby, H A Bogard, 1996, Metered-dose inhalers 1. Drug content and particle size distribution of beclomethasone dipropionate: Journal of Pharmaceutical and Biomedical Analysis, v. 14, p. 793-800.
Lewis,D, S Johnson, B J Meakin, D Ganderton, G Brambilla, R Garzia, P Ventura. Effects of Actuator Orifice diameter on beclomethasone dipropionate delivery from a pMDI BFA solution formulation. VI, 363-364. 1998. Respiratory Drug Delivery. Ref Type: Conference Proceeding
Prinn,K B, H R Costantino, M Tracey, 2002, Statistical Modeling of Protein Spray Drying at the Lab Scale: AAPS PharmSciTech, v. 3, p. 1-8.
Quan,C P, S Wu, N Dasovich, C Hsu, T Patappff, E Canova-Davis, 1999, Susceptibility of rhDNase I to glycation in the dry-powder state: Analytical Chemistry, v. 71, p. 4445-4454.
Quinn,E A, R T Forbes, A C Williams, M J Oliver, L McKenzie, T S Purewal, 1999, Protein conformational stability in the hydrofluoroalkane propellants tetrafluoroethane and heptafluoropropane analysed by Fourier transform Raman spectroscopy: International Journal of Pharmaceutics, v. 186, p. 31-41.
Sonie,W H, F E Blondino, P R Byron, 1992, Chemical Stabiliy pressurised inhalers formulated as solutions: Journal of Biological and Pharmaceutical Science, v.3, p. 41-47.
Stahl,K, M Claesson, P Lilliehorn, H Linden, K Backstrom, 2002, The effect of process variables on the degradation and physical properties of spray dried insulin intended for inhalation: Intemational Journal of Pharmaceutics, v. 233, p. 227-237.
Stein,S W, 1999, Size distribution measurements of metered dose inhalers using Andersen Mark 11 cascade impactors: International Journal of Pharmaceutics, v. 186, p. 43-52.
Vervaet,C, P R Byron, 1999, Drug-surfactant-propellant interactions in HFA-formulations: International Joumal of Pharmaceutics, v. 186, p. 13-30.

1. A formulation of a therapeutic substance suitable for delivery to a patient by a metered dose inhalation device, the formulation comprising a substantially dry powder preparation of the substance in association with a stabilising amount of a carbohydrate compound or derivative thereof and a polyhydroxylated polyalkene in combination with one or more propellants therefor, wherein the therapeutic substance is selected from peptides and proteins.

2. A formulation according to claim 1, further comprising a cosolvent for said substance.

3. A formulation according to claim 1, wherein the therapeutic substance is selected from antibodies, interferons, enzymes, hormones, euprolide acetate, CFTR, and α1-antitrypsin.

4. A formulation according to claim 3, wherein the therapeutic substance is a hormone selected from insulin, LHRH, granulocyte-colony stimulating factor, calcitonin, heparin, human growth hormone, and parathyroid hormone.

5. A formulation according to claim 1, wherein the substance is dnase I.

6. A formulation according to claim 1, which is non-immunogenic.

7. A formulation according to claim 1, which is capable of being stored at room temperature without losing more than 50% biological activity of the therapeutic substance after two months.

8. A formulation according to claim 1, wherein the carbohydrate compound comprises at least one oligosaccharide.

9. A formulation according to claim 8, wherein the carbohydrate compound comprises at least one disaccharide.

10. A formulation according to claim 9, wherein the disaccharide is selected from trehalose, mannitol, sucrose, and mixtures thereof.

11. A formulation according to claim 1, wherein the carbohydrate compound or derivative thereof constitutes between about 30% and 400% by weight of the therapeutic substance.

12. A formulation according to claim 1, wherein the propellant is alkane based.

13. A formulation according to claim 12, wherein the propellant is at least one haloalkane.

14. A formulation according to claim 13, wherein the propellant is selected from HFA-134a and HFA-227.

15. A formulation according to claim 1, wherein at least one polyhydroxylated polyalkene has the general structure —(CH₂—CHOR)ₙ— where R is the same or different from one monomeric unit to the next, and is hydrogen, lower alkyl, lower alkenyl, lower alkanoyl, lower alenoyl or is a bridging group between adjacent monomers.

16. A formulation according to claim 15, wherein, when R is not hydrogen, the number of carbon atoms, excluding any —CO— group, is between 1 and 6, inclusive.

17. A formulation according to claim 15, wherein the polyhydroxylated polyalkene is selected from polyvinylalcohol, polyvinylacetate, polyvinyl alcohol-co-vinyl acetate, poly(vinyl butyral), poly(vinyl alcohol-co-ethylene), and mixtures thereof.

18. A formulation according to claim 17, wherein the polyhydroxylated polyalkene is PVA.

19. A formulation according to claim 18, wherein the PVA a hydrolysate of PVAc, the level of hydrolysis being between 40% and 100%.

20. A formulation according to claim 18, wherein the PVA a hydrolysate of PVAc, the level of hydrolysis being between 50 and 90%.

21. A formulation according to claim 18, wherein the PVA has a molecular weight of between about 9 kDa and 50 kDa.

22. A formulation according to claim 1, wherein the polyhydroxylated polyalkenes are present in an amount of from about 5% to about 200% by weight of the therapeutic substance.

23. A formulation according to claim 22, wherein the polyhydroxylated polyalkene is present between about 10% and about 50% by weight of the substance.

24. A method for the preparation of a formulation as defined in claim 1, comprising blending the therapeutic agent with the carbohydrate compound or derivative thereof and polyhydroxylated polyalkene substances in an aqueous vehicle, drying the resulting blend to a powder, and then formulating with propellant.

25. A method according to claim 24, wherein the aqueous vehicle is selected from saline, a suitable buffer, and deionised water.

26. A method according to claim 24, which comprises spray—drying the blend.

27. A powdered formulation of a therapeutic agent, a carbohydrate compound or derivative thereof and a polyhydroxylated polyalkene, as defined in claims 1, which is suitable for incorporation with a haloalkane propellant for dispensing from a metered dose inhaler.

28. A powdered formulation according to claim 27, wherein the powder particles have an aerodynamic diameter of between about 1 μm and 50 μm.

29. A metered dose inhalation device provided with a reservoir comprising a formulation according to claim 1.
